(19) United States
(12) Patent Application Publication
(10) Pub. No.: US 2026/0325321 A1
(43) Pub. Date: Oct. 22, 2026

(54) MYOELECTRIC-CONTROLLED PROSTHETIC DEVICE AND METHOD FOR CALIBRATION AND USE OF SAID DEVICE

(71) Applicant: BionIT Labs S.r.L., Soleto (LE) (IT)

(72) Inventors: Chiara Macri', Soleto (LE) (IT); Matteo Aventaggiato, Soleto (LE) (IT)

(73) Assignee: BionIT Labs S.r.L., Soleto (LE) (IT)

(21) Appl. No.: 19/723,376

(22) Filed: Jun. 29, 2026

Related U.S. Application Data
(63) Continuation of application No. 18/029,476, filed on Sep. 2, 2021.

(30) Foreign Application Priority Data
Sep. 30, 2020 (IT) 102020000023011

(51) Int. Cl. A61F 2/72; A61F 2/70; A61F 2/76

(52) U.S. Cl. CPC A61F 2/72; A61F 2002/704; A61F 2002/763; A61F 2002/7665

(57) ABSTRACT

A prosthetic device comprising: a limb or artificial joint or orthosis or exoskeleton comprising a mechanism provided with at least an actuator and configured to carry out one or more actions; a supplying source; at least an input source modulated by the contraction of one or more muscles of the subject wearing the device, comprising at least an electrode; electronic computing means, characterized in that the computer programs are loaded configured to carry out the method comprising the steps of:
recording the signals (Ns) detected
subdividing each of said signals
extracting from the signal relative to each of the time intervals
calculating a statistical estimator for each feature calculated
repeating the steps
associating a vector defined by the set of the values of the features calculated.

### RELATED APPLICATIONS

This is a Continuation-In-Part of U.S. application Ser. No. 18/029,476 now abandoned. U.S. application Ser. No. 18/029,476 was a U.S. National Stage Application of PCT application PCT/IB2021/058004 having an international filing Date of 2 Sep. 2021. This present application claims foreign priority based on application No. 102020000023011 of Italy, filed on 30 Sep. 2020.

### BACKGROUND OF THE INVENTION

### 1. Field of the Invention

The present invention relates to a myoelectric-controlled prosthetic device and a method for calibration and usage of said device.

It is to specified that in the present document the term prosthetic device refers to a device comprising a prosthesis, an orthosis, a partial or integral exoskeleton and relative control means.

In particular, the present invention relates to a myoelectric-controlled prosthetic device to be used with a particularly rapid and intuitive training of the patient, and not subject to physiological variations of the signal levels detected by the myoelectric sensors over the time.

### 2. Brief Description of the Prior Art

People born with a limb, and who, during their life, lose it in an accident or due to an illness, often keep the capacity to contract the muscles which articulated the limb previously. On the contrary, people born without a limb due to a genetic malformation or other neonatal illnesses or accidents have no feeling of “ghost limb” but can imagine anyway the movements the failing limb could carry out after determined muscle contractions thanks to the experience of the contralateral limb. There exists also a group of people who, owing to neuromuscular disorders, absence of stimulations from the environment or other physiological/environmental reasons, have not matured the expectation of a proprioceptive feedback of the muscular contraction effects on the amputated or missing limb.

According to what known at the state of the art, a myoelectric prosthetic device comprises:
a limb or artificial joint or exoskeleton or orthosis comprising a mechanism provided with at least an actuator and configured to carry out one or more actions (for example a prosthetic hand can close and open, an exoskeleton arm can support the natural movement of the limb);
a supplying source (for example an electric battery);
at least an input source (and preferably more than one input source) modulated by the contraction of one or more muscles of the subject wearing the device (for example one or more electrodes for detecting surface muscular potentials, called surface electromyographic signals, sEMG);
electronic computing means, on which computer programs are loaded, configured to detect the signals coming from said at least one input source and to actuate said at least one actuator in order that said device carries out an action as a function of the signals coming from said at least one input source.

According to embodiments known at the state of the art, the prostheses are directed to all the just described categories of amputees, with three possible approaches described in the following: in case of residual hypotrophic muscles, absence of space for positioning more than one electrode, antagonist muscles innervation loss, only one electrode is applied at one of the residual muscles used to activate the amputated or missing limb, and the prosthesis is actuated by carrying out two movements thanks to the setting of two thresholds on the amplitude of the sEMG signal measured for the unique electrode provided. For example, in case of prosthetic hand, this can open when the first threshold is reached, and close when the second threshold is reached, as it is described for example in Merletti at al Electromyography: Physiology, Engineering, and Non-Invasive Applications, 2004, IEEE Press Series on Biomedical Engineering.

In other cases, two electrodes are applied, one being positioned at agonist muscles and one at antagonist muscles. In this case, an actuation threshold is set for each electrode to control the movement of agonist/antagonist muscles. For example, in case of prosthetic hand the activation of the finger flexor muscles activates the hand closing and the activation of finger extensor muscles activates its opening).

Anyway, in this configuration it is needed to set a biunivocal correspondence between electrode and controlled movement. In the prosthetic hand example, the electrode positioned on the flexor muscles controls the closing movement while the electrode positioned on the extensor muscles controls the opening movement.

At the state of the art, for example in U.S. Pat. No. 10,448,857B2 and U.S. Pat. No. 9,566,016B2, prostheses are known that comprise a crown of three or more electrodes. In these systems, the electrodes have to be arranged with a precise order since the amplitude of the drawn sEMG signals can be used to pilot multi-parametric systems, such for example systems based on Kiviat diagrams whose axes represent the amplitude of each channel. In these cases, the subject is normally subjected to a difficult training step, during which he learns to carry out constantly the contractions needed for the generation of correct polygons of the Kiviat diagram which enable the prosthesis correct movements.

In the calibration of a prosthesis, it is also needed to calibrate the torque and/or the prosthesis activation speed. The most intuitive case is the one of a robotic hand, in which the activation torque determines the strength with which the hand grabs an object.

According to the calibration methods known at the state of the art, an EMG relation vs strength or muscle torque is imposed, possibly derived from the contemporaneous recording of the torque exerted by the hand joints of a healthy subject, and the electromyographic signals detected by the electrodes installed on the limb of such subject. Such relation between measured strength or torque and electromyographic signals is used to build a calibration curve used then for the prostheses worn by amputees. These relation curves EMG-strength or EMG-torque, when pre-determined by literature studies and applied directly on amputees, are little efficient since this kind of solution does not consider the subject muscle morphology and his anthropometric characteristics and, so, it is likely to not allow the subject to use the prosthesis instinctively and immediately.

Document US2016278947 describes a prosthetic system comprising computing means configured to acquire signals from an angular velocity sensor and to control the prosthesis as a function of these acquired signals.

### Technical Problem

All the devices known at the state of the art and in particular the methods that such devices use to associate the movements of the prosthetic device with the signals detected by the input sources are limited, since:
if to activate the prosthesis movements thresholds on the sEMG signals amplitude are used, it is often needed to adapt them periodically upon physiological variations the signals detected over the time are interested by. In fact, it is known that weather variations and the physical activity carried out by the subject modify the skin microcirculation characteristics, the sweating and so the bioelectric characteristics of the electrode-skin coupling. The electro-skin impedance change leads to a change of the sEMG signal amplitude characteristics and so, the set thresholds are reached with higher or lower difficulty with the same muscle contraction carried out. So, this requires periodical interventions by the orthopedic technician for adjusting the threshold values,
if to activate the prosthesis movement, pattern recognition techniques are used by means of supervised machine learning procedures, the settings are to be modified over the time owing to the electrode-skin contact impedance variations or owing to muscle tissues physiological variations causing intensity variations of the detected signals. In fact, the usage of myoelectric prostheses leads the amputee or the subject born with a malformation to contract more often the muscles used to pilot the prosthesis. These contractions act as a sort of muscle training which manifests itself both as an increase in tissue volume and as more ability and capacity of the subject to contract such muscles selectively.

Another problem is to calibrate the torque and/or activation speed of a prosthesis or an orthosis worn by amputees who have only residual muscles or were even born without any limbs. In these subjects the calibration has to be carried out without any kind of feedback and by subjects who have never known, in their life, the feeling of moving the limb substituted by the prosthesis.

Generally, it is needed to guarantee stability to the movement recognition algorithm. Stability means the capacity of an algorithm to recognize the instruction expressed by the subject with low error, without often ending in error situations which could lead to the impossibility to manage/pilot the prosthesis. This is a condition potentially developable by all the systems integrating machine learning algorithms (such for example fuzzy decisional algorithms or neuronal nets, in particular not linear ones). These results are not reached by the calibration methods known at the state of the art.

### SUMMARY OF THE INVENTION

So, the present invention provides a prosthetic device which overcomes the limits linked to the embodiments known at the state of the art, and in particular which eliminates the need of configuration for the user or technician, which does not require the association of each electrode with a specific movement, which can be used indifferently with any number of electrodes since the number of connected electrodes does not increase the configuration or calibration complexity.

Yet, the present invention provides a prosthetic device able to adapt autonomously and automatically its own functioning to the final user physiologic characteristics, and which is able to adapt its own configuration parameters automatically over the time, so that a correct functioning is kept even while the user physiological conditions vary or while the environmental conditions in which the device is used vary, without any intervention of the technician or final user.

Yet, the invention provides a prosthetic device able to pilot the torque/speed of one or more movements proportionally to the muscle contraction and, in particular, which is able to carry out rapidly and intuitively, also for subjects with only residual muscles, or who have never had in life the feeling to move the limb substituted by the prosthesis, the calibration of the torque and/or activation speed. Yet, the invention provides a prosthetic device configured to store over time coefficients, feature values, statistical estimators, reference vectors, calibration parameters, control parameters and/or contextual parameters, and to process said stored data in order to generate statistical information, classify operating or use conditions, support recalibration, maintenance or technical assistance activities, provide information representative of the ability of the subject to use the device, and/or generate training data for data-driven control models.

### BRIEF DESCRIPTION OF THE DRAWINGS

FIG. 1 is a schematic representation of the system where is shown a prosthetic limb with multiple input source placed on a human forearm. Said prosthetic limb is connected to a processing unit which controls an actuator inside the prosthetic hand.

FIG. 2 is a perspective view of a prosthetic device according to an embodiment of the invention, showing an array of input sources in a crown configuration on the forearm.

FIG. 3 is a flowchart illustrating the supervised calibration procedure, including steps for signal acquisition 100, segmentation into time windows 110, feature extraction 120, and the use of a statistical estimator 130 to generate a feature vector output.

FIG. 4 is a graphical representation of a K-dimensional feature space, depicting clusters of feature vectors and their respective boundaries relative to coordinate axes, where the highest cluster represents the rest condition, while the rest of the clusters represent different movements.

FIG. 5 is a flowchart diagram representing supervised calibration process, sequentially showing signal acquisition 100, segmentation 110, feature extraction 120, statistical processing 130, and the final generation of vectors.

FIG. 6 and FIG. 7 are schematic diagrams illustrating the supervised calibration procedure for discrete limb orientations, showing the device on a limb in an “UP” and “DOWN” orientation to generate corresponding calibration sets “A” and “B”.

FIG. 8 is a flowchart depicting the steps top perform the test 2, comprising the steps of acquiring the input signal 200, extracting the features 210, comparing said features with stored vectors and select the closest match 220, output movement 230.

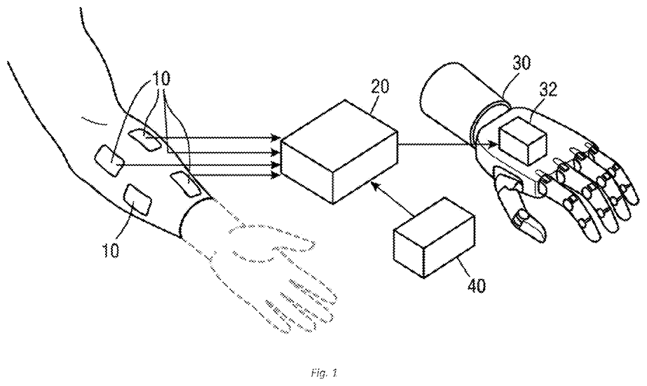

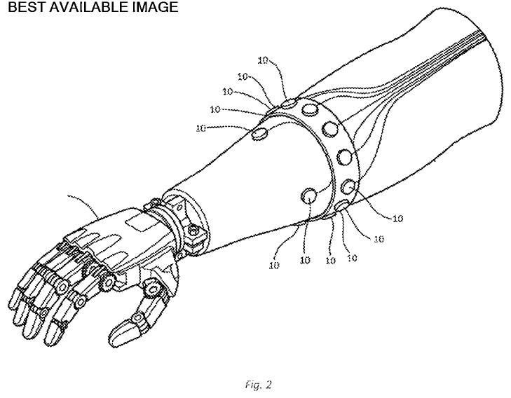

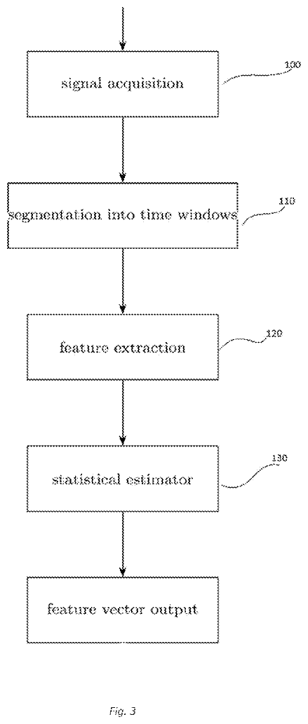

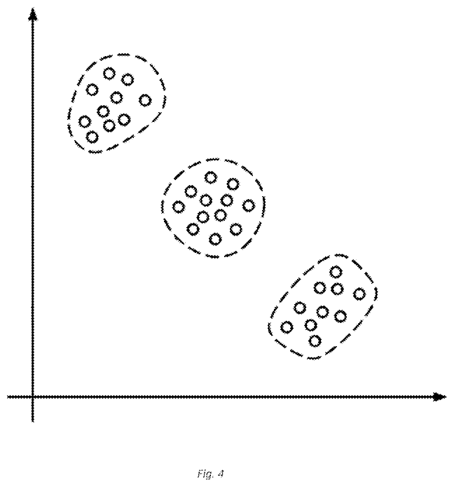

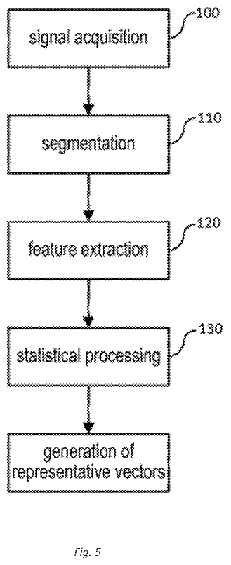

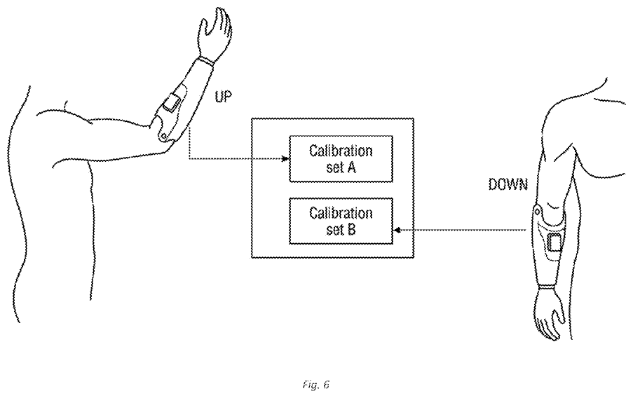

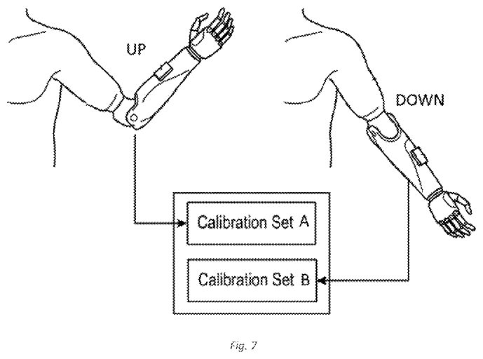

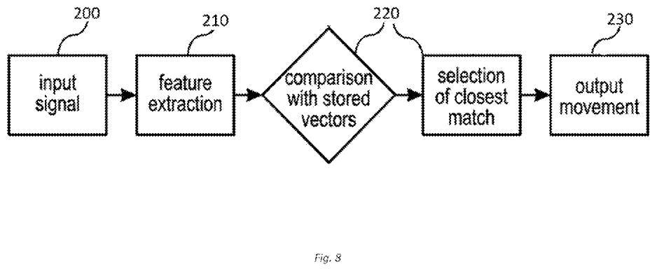

### DETAILED DESCRIPTION OF THE PREFERRED EMBODIMENTS

The prosthetic device according to the invention comprises:
a limb or artificial joint (or an exoskeleton or orthosis) comprising a mechanism provided with at least an actuator and configured to carry out one or more actions;
a supplying source (for example an electric battery);
at least an input source (and preferably more than one input source) modulated by the contraction of one or more muscles of the subject wearing the device (for example one or more electrodes for detecting surface muscular potentials, called surface electromyographic signals, sEMG);
electronic computing means, on which computer programs are loaded, configured to detect the signals coming from said at least one input source and to actuate said at least one actuator in order that said mechanism carries out an action as a function of the signals coming from said at least one input source.

In one exemplary embodiment, the prosthetic device comprises a prosthetic limb portion, such as a prosthetic hand or other terminal device, mechanically coupled to a socket or support structure configured to be worn by the subject. The prosthetic limb portion comprises one or more movable members, for example one or more fingers or grasping elements, which are mechanically driven by at least one actuator, such as an electric motor, a geared motor, a linear actuator, a tendon-driven mechanism, or an equivalent electromechanical actuation unit.

The actuator is operatively connected to an electronic control unit of the prosthetic device. The electronic control unit may comprise one or more processors, microcontrollers, memories, signal conditioning circuits, analog-to-digital converters, motor drivers and communication interfaces. The electronic control unit is configured to receive input signals from one or more input sources, preferably myoelectric electrodes placed on the residual limb or on another body portion of the subject, process said input signals according to the method described herein, and generate one or more control signals for the actuator.

In use, the contraction of one or more muscles of the subject generates electrical signals that are detected by the input sources. These signals are acquired by the electronic control unit, processed to identify the movement intended by the subject, and converted into actuator control commands. The actuator then causes the prosthetic mechanism to carry out the corresponding movement, for example opening, closing, gripping, releasing, flexing, extending, rotating or otherwise moving a prosthetic component.

The device comprises also preferably:
at least a sensor, and preferably a plurality of sensors, integrated in the system and configured to detect information about the status of the device or its components. For example, there can be provided one or more temperature sensors, one or more inertial units for detecting the relative position in the space of the various components of the device, one or more pressure sensors for detecting the contact with outer objects;
a mechanic protection against the outer environment (for example a cosmetic glove in impermeable and/or shockproof material, in case of a transradial prosthesis).

The specific mechanical implementation of the prosthetic mechanism is not limiting. The actuator may drive the movable prosthetic member through gears, tendons, linkages, belts, screws, cams, compliant mechanisms or any other suitable transmission arrangement. Similarly, the prosthetic device may comprise a prosthetic hand, a hook, a wrist unit, an elbow unit, a terminal device, an orthosis or an exoskeleton portion, provided that the electronic computing means are operatively connected to at least one input source and to at least one actuator so as to control a physical movement of the device.

The prosthetic device according to the invention is characterized in that, on said electronic computing means computer programs are loaded configured to carry out the method described in the following. The method according to the invention comprises the steps of:
(0) checking the presence of calibration parameters in the memory;
   in affirmative case, carrying out a calibration test procedure (2), in negative case, carrying out a supervised calibration procedure (1), after which carrying out the calibration test procedure (2);
   at the result of the calibration test procedure (2), in positive case going to the device using condition (3) and not supervised calibration (4), in negative case repeating the supervised calibration.

In order to carry out the supervised calibration procedure (1), the method comprises the next steps.
(100) recording the signals (Ns) relative to each electrode provided in the system with the user in a predetermined condition (rest condition or condition of execution of a predetermined movement) for a predetermined time interval ΔT, thus obtaining N tracks or—pieces—of electric signal in the time domain, one relative to each sensor.

It is to be specified that rest condition means a condition in which the subject is asked to remain still, with relaxed muscles, in an inactivity state, thus avoiding contracting muscles, avoiding exerting pressure on the portions of the prosthetic device and avoiding activating possible sensors provided in the system. Said predetermined time interval, according to the kind of connected electrodes and the kind of prosthetic actuator, can be varied, for example, between 1 and 10 seconds.

(110) subdividing each of said signals (Ns) in a plurality of time intervals (also called epochs or windows) of predetermined duration, thus obtaining “M” signal pieces for each signal. The duration of each window, according to the kind of signals and the kind of prosthetic actuator, is preferably between 200 ms and 2 seconds. It is also to be specified that the time windows can be partially overlapping or not. The overlapping allows to have a greater number of windows at the same signal duration, thus maximizing the extraction of information contained therein, also in presence of noise;
(120) for each one of the M windows of each recorded signal, extracting at least a characteristic parameter (feature) relative to the signal in the time domain, or relative to the signal FFT transform. The extracted features comprise preferably one or more of the following ones: peak or peak-to-peak average amplitude, peak or peak-to-peak maximum amplitude, effective value, power spectrum average frequency, power spectrum median frequency, −6 dB power spectrum band, −20 dB power spectrum band. In this way, it is obtained a matrix F=K×M, wherein K is the number of computed features, M is the number of epochs and each element F(i,j) of the matrix is the value of the i^{th} feature calculated on the j^{th} signal window;
(130) calculating a statistical estimator of the value of each feature calculated at point (120). In a preferred embodiment, said statistical estimator is the median M; in another embodiment said statistical estimator is the signal average. In this way, it is obtained a vector of dimensions K×1, wherein K is the number of feature considered. The vector defines the coordinates of a point representing the rest condition (rest) or the predetermined movement of point (100) in a K-dimensional space, each dimension being relative to a signal feature.

The above-described procedure can comprise one or more of the following variations:

Possibly at point (110), both in case of acquisition of the values relative to the rest position and in case of acquisition of the values relative to a predetermined movement, the starting portions of the recorded signals (for example a portion between 5 and 20%, according to the recording conditions) can be discarded to be certain that the subject is really in the condition of rest or of execution of the predetermined movement, since in the starting step the subject cannot be in the required condition yet. Possibly after point (130), the point can follow of (131) calculating, for each feature, the standard deviation of the values distribution, comparing each feature with its own statistical estimator, and discarding the values with a difference—in absolute value—greater than twice the standard deviation from said statistical estimator.

Concerning the acquisition of signals relative to a predetermined movement, it is to be specified that the kind of muscle contraction to be associated with the movement actuated by the mechanism of the device can be different from the kind of contraction a not amputee would carry out for carrying out the same movement.

For example, a transhumeral amputee has not the forearm muscles that he would normally use to activate the finger flexing, but he could use the flexor muscles of the forearm (one or two heads of the biceps brachii). Similarly, a trans-radial amputee, even if he has still the muscles used for the natural flexion of the fingers, in order to optimize the piloting of the movement relative to the finger flexion could prefer to carry out the muscular contraction associated with the wrist flexion, since the muscular group contracting in this case is mainly concentrated on the inner side of the forearm, thus exposing the detecting electrodes to a lower cross-talk phenomenon. Similarly, in case of opposite movement (in the example the finger extension), a trans-humeral amputee has not the forearm muscles that would normally use to activate the finger extension, but he can use the extensor muscles of the forearm (one or more heads of the triceps brachii). Similarly, a trans-radial amputee, even if he has still the muscles used for the natural finger extension, in order to optimize the piloting of the movement relative to the finger flexion can prefer to carry out the muscular contraction associated with the wrist extension, since the muscular group contracting in this case is mainly concentrated on the outer side of the forearm, thus exposing to a lower cross-talk phenomenon.

The steps (100) to (131) are carried out sequentially for a rest condition (rest) and for a plurality of predetermined movements (Mov_1, Mov_2, . . . , Mov_n), thus obtaining a plurality of vectors of dimensions K×1 defining each, in the K-dimensional space relative to the K acquired features, the position of a point representing each detected movement.

The method provides preferably the execution of the above steps sequentially for a movement and in the following for the movement opposite thereto. For example, in case of trans-radial prosthesis, if the first calibrated movement (Mov_1) is the finger flexion, the second movement (Mov_2) is normally the finger extension.

Moreover, preferably, at the end of the calibration procedure for all the movements intended, the calibration of the rest condition (rest) is carried out again in order to compensate possible alterations of the sEMG signal features upon the introduction of muscular fatigue phenomena. In order to do so, the steps (100) to (131) for the rest condition are carried out, thus obtaining the definition of another point representing the rest condition in K-dimensional space. The point representing the rest condition is then calculated as the average point between the first and the second point calculated for said condition.

The rest condition can be also object of detection before and after the detection relative to each movement, by calculating for each of the K features relative to the rest condition the average value of all the values calculated for each detection, in order to define the point representing the rest condition in the K-dimensional space.

According to a preferred embodiment, the prosthetic device according to the invention comprises at least a position sensor, configured to detect the position of said device, and is configured to store for each movement, as well as for the rest condition, a plurality of reference points and to select automatically the suitable reference point to be considered during usage as a function of the position of said prosthetic device detected by said position sensor. Said position sensor is preferably an inertial unit (IMU, inertial measuring unit).

This characteristic satisfies the need to compensate possible alterations of the signals recorded by the sensors due to the position variation of the prosthetic device. For example, in case of a trans-radial prosthesis piloted by one of more EMG sensors, the variation of the arm position can cause the variation of the relative position electrode-muscle even in absence of variation of the relative position electrode-skin. In this case, by using an IMU positioned in the storage (integrated or not in the electrodes, or in the storage itself or in the battery housing, or in other components of the prosthesis), it is possible to recognize the position of the prosthesis and, on the basis thereof, to select automatically and to use the most suitable calibration parameters.

In order to calibrate the functioning of the device in a plurality of positions, the supervised calibration procedure (1) comprises the steps of:
(80) defining a plurality of significant positions (P₁, . . . , Pᵣ);
(85) making the user wearing the device assume the first of said positions (P1);
(90) carrying out the steps (100) to (131), while storing a first set of reference points relative to each movement carried out with the device in said first position (P1);
(95) repeating the steps (85) and (90) for all the positions (P₁, . . . , Pᵣ) defined at point (80) while storing for each one thereof a relative set of reference points relative to each movement carried out with the device in said position. After the execution of the supervised calibration procedure, in order to increase the calibration stability (i.e. in order to reduce the error frequency), the method can provide the execution of the following steps:
(140) calculating the distance in the K-dimensional space between the point representing each kind of movement or rest and all the other points representing further conditions of movement or rest.

It is to be specified that in a first embodiment the distance of point (140) can be calculated as Euclidean distance in a K-dimensional space; in a second embodiment at each dimension of K-dimensional space a specific “weight” can be assigned, and the distance can be calculated as the Euclidean distance in the K-dimensional space in which each addend of the summation of the squares of the distances along each axis is multiplied by its own weight.
(150) comparing said distances calculated at point (140) with a minimum threshold of predetermined distance;
(160) in case of presence of points representing the movements distant from the points representing the other movements less than the threshold of point (150), individuating said movements and sending a warning message to the user;
(170) as a function of a user decision, carrying out again the supervised calibration procedure or eliminating one or more movements selected by the user among the ones individuated at point (160).

In this way, it is obtained the definition of a series of points representing a plurality of movements distant to each other more than the minimum threshold. This reduces the possibility of error in the next step of device usage, as it will be clearer in the following.

Once the supervised calibration step according to the just described procedure is ended, the method provides the possibility to carry out a test (2) of the recorded parameters, according to the following steps:
(200) detecting the parameters relative to the signals of each electrode while the user carries out one specific movement,
(210) calculating, with the same modes described in steps (110) to (130), the coordinates of the point representing the movement carried out by the user.
(220) individuating, among all the points representing the movements (or the rest condition) stored during the supervised calibration procedure, the one with the lower distance from the point defined at step (210).
(230) providing the user with a feedback indicating the movement individuated at point (220).

It is to be specified that in a first embodiment, the distance of point (220) can be calculated as Euclidean distance in a K-dimensional space; in a second embodiment at each dimension of the K-dimensional space a specific “weight” can be assigned, and the distance can be calculated as the Euclidean distance in K-dimensional space in which each addend of the summation of the squares of the distances along each axis is multiplied by its own weight.

So, the user can consider the test procedure as successful in case the feedback of point (230) indicates the movement he was really carrying out.

In case the recognition of movements is not satisfying, the user can repeat the supervised calibration procedure (1), by repeating it as a whole or only for some movements (or for the “rest” condition). In case, instead, the configuration obtained with the supervised calibration is satisfying, it is validated and used for the next steps.

Clearly, in case a plurality of sets of reference points relative to a plurality of significant positions of the device are provided, the test procedure (2) can be conveniently carried out for each of said significant positions.

It is to be specified that the feedback provided at step (230)—as also the warning of step (160)—can be:
a. a visual feedback of text kind through a user interface showing the recognized movement with a message (“rest”, “movement 1: hand closing”, “movement 2: hand opening”, etc.).
b. a visual feedback on a software user interface connected to the system which, through the virtual representation in 2D or 3D of the prosthesis or orthosis or exoskeleton used, shows in real time in animated or static way how the actuated movement would be.
c. visual feedback of a dynamic histogram, where each bar relates to one of the features analyzed on the inputs and it varies its own value as a function of the measured intensity;
d. acoustic feedback where each feature analyzed on the inputs modulates a “note” through the modulation both in amplitude and frequency of a carrier frequency as a function of the value assumed by the relative feature;
e. visual feedback provided by the same prothesis or orthosis or exoskeleton by means of the actuation of the real movement intended.
f. haptic feedback.

Moreover, preferably, at step (230), the device is configured to provide the user with an indication relative to the distance between the point calculated at step (210) and the point individuated at step (220). The indication can be provided by means of various feedback tools:
a. visual feedback of text kind through a graphic interface showing a numeric indication (percentage or absolute) of the correspondence between the instruction expressed and the movement selected as target;
b. visual feedback on a software user interface connected to the system which, through the virtual representation in 2D or 3D of the prosthesis or orthosis or exoskeleton used, shows in real time the effective recognition of the movement selected thorough animated or static images;
c. visual feedback of a dynamic histogram, where each bar relates to one of the features analyzed on the inputs and it varies its own value as a function of the measured intensity; in particular, it is provided a reference (for example a line or other type of marker on the same bar of the histogram) highlighting the target intensity level for each of the analyzed features;
d. acoustic feedback where each feature analyzed on the inputs modulates a “note” through the modulation both in amplitude and in frequency of a carrier frequency as a function of the value assumed by the relative feature; in order to provide another feedback, the amplitude of the sounds can increase while approaching the target combination for the selected movement;
e. visual feedback provided by the same prothesis or orthosis or exoskeleton by means of the actuation of the same movement selected.

After the calibration test procedure (2), in positive case, the method provides to carry out the procedure of usage of the device (3) and the not supervised calibration (4).

The procedure of usage of the device (3) provides the steps of:
(300) acquiring, at predetermined time intervals, the signals (Ns) coming from said electrodes for a predetermined duration and storing them in a relative buffer.

Said predetermined time intervals are preferably between 200 ms and 1000 ms; said predetermined duration is preferably between 200 ms and 2000 ms. (310) subdividing each of said signals (Ns) in a plurality of time intervals (also called epochs or windows) of predetermined duration, thus obtaining “M” signal pieces for each signal. The duration of each window, according to the kind of signals and the kind of prosthetic actuator, is preferably between 200 ms and 2 seconds. It is also to be specified that the time windows can be partially overlapping or not. The overlapping allows to have a greater number of windows at the same signal duration, thus maximizing the extraction of information contained therein, also in presence of noise.
(320) for each one of the M windows of each recorded signal, extracting at least a characteristic parameter (feature) relative to the signal in the time domain, or relative to the signal FFT transform. The features extracted during the using step are the same features extracted in the calibration step at step (120),
(330) calculating a statistical estimator of the value of each feature calculated at point (320). The calculated statistical estimator has to be the same used in the calibration step at point (130). In this way, it is obtained a vector of dimensions K×1, wherein K is the number of features considered. The calculation modes adopted, including the discarding of the values distant from the statistical descriptor, are the same used in the supervised calibration step.
(340) calculating the Euclidean distance between the point calculated at step (330) and each point representing each movement stored during the supervised calibration procedure, thus identifying the movement for which such distance is minimum.
(350) carrying out the movement identified at step (340).

It is clear that, generally, the word “movement” can mean also the “rest” condition.

The above method is preferably executed by the electronic computing means of the prosthetic device in cooperation with the hardware components of the device. More specifically, the input source generates analog or digital signals representative of a physiological activity of the subject, such as surface electromyographic signals. If analog signals are generated, the electronic control unit may condition, amplify, filter and convert the signals into digital samples by means of suitable signal conditioning circuitry and analog-to-digital conversion circuitry.

The digital samples are stored in a memory buffer and are segmented by the processor into time windows of predetermined duration. For each time window, the processor calculates one or more features representative of the acquired signal. The calculated features are then used to define a feature vector in a multidimensional feature space. During calibration, feature vectors associated with a rest condition and with one or more intended movements are stored in memory as reference vectors or calibration data. During subsequent use of the prosthetic device, the electronic control unit acquires new signals from the input sources, calculates corresponding feature vectors in real time or near real time, and compares said feature vectors with the stored reference vectors. Based on the result of the comparison, the electronic control unit selects the movement associated with the closest or otherwise most suitable stored vector and generates a corresponding actuator command.

The generated actuator command may comprise, for example, a motor enable signal, a direction signal, a speed command, a torque command, a pulse-width modulation signal, a current command, a position command or another electrical control signal suitable for driving the actuator. Therefore, the software processing steps directly determine the electrical command supplied to the actuator and consequently the physical movement performed by the prosthetic mechanism.

The method is not intended to be performed independently of the prosthetic device hardware. The acquisition of physiological signals, the generation of sampled signal data, the segmentation of said sampled signal data into time windows, the extraction of features from said sampled signal data, the comparison with stored calibration data and the generation of an actuator command are performed in the context of the operation of the prosthetic device. The calculated output is not merely an item of information presented to a user, but is used by the electronic control unit to control the actuation of the prosthetic mechanism.

Accordingly, the processing performed by the electronic computing means produces a technical effect on the prosthetic device, namely the selection and execution of a physical movement of the prosthetic mechanism in response to signals generated by the subject. The method therefore cooperates with the electrodes, the electronic control unit, the memory and the actuator to improve the control of the prosthetic device.

Moreover, in case a plurality of sets of reference points relative to a plurality of significant positions (P1, . . . , Pᵣ) are provided, the procedure of usage of the prosthetic device (3) comprises, before the step (300), the steps of:
(298) acquiring by means of said position sensor the position of the device, thus identifying the significant position of reference;
(299) selecting the set of reference points representing each movement relative to the position identified at point (298). The above-described procedure can comprise one or more of the following variants:
   the values of the medians can be substituted with the average values or of other statistical estimator according to the kind of feature considered;
   the starting portions of the recorded signals (for example a portion between 5 and 20%, according to the recording condition) can be discarded to be certain that the subject is really able to express the instruction of possible movement in a stable condition.

Moreover, in order to reduce the errors of rest condition recognition, defined as D_{MOV} the distance of the point individuated at step (330) from the point representing the movement identified at step (340) and as D_{REST} the Euclidean distance of the point individuated at step (330) from the point representing the rest condition, at step (350) the following condition can be added:
if D_{MOV}≤α·D_{REST} carrying out the movement identified at step (340),
if D_{MOV}>α·D_{REST} keeping the device in rest condition.

Wherein α is a suitable coefficient equal or lower than 1 and greater than 0. In particular, the more a is low, the lower the system is sensible to the actuation of movements; for too low values of a the system could not recognize any movement. Alfa is preferably between 0.75 and 0.95.

It is now described the procedure “not supervised calibration” (4).

As yet said, this procedure allows to consider automatically and in absolutely transparent manner for the user, all the variations over the time due to factors such as: variations of the impedance of the electrode-skin contact, physiological variations of the muscle tissues due to fatigue, muscle training or vascularization change, alterations of signals recorded by sensors sensible to environmental conditions.

More specifically, this not supervised calibration procedure (4) intervenes automatically to modify, if needed, the reference points in the multidimensional space relative both to the rest condition and to each movement.

The procedure provides the following steps:
(400) with the prosthetic device used, starting a timer (countdown) of predetermined period.

Said period is preferably between 60 seconds and 30 minutes.
(410) storing the values of the features relative to the first movement following the timer expiring of point (400) associated with the rest condition, thus defining the point Rest_1 in the K-dimensional space, and starting a new timer of predetermined period.
(420) storing the values of the features relative to the first movement following the timer expiring of point (410) associated with the rest condition, thus defining the point Rest_2 in the K-dimensional space.
(430) calculating the Euclidean distances of the two points defined at steps (410) and (420) from the reference point representing the rest condition (Rest_ref).
(440) In case the distance from the reference point (Rest_ref) of the point (Rest_2) defined at step (420) is greater than the distance from the reference point (Rest_ref) of point (Rest_1) defined at step (410), updating the values of the reference point, on the contrary, not updating said values.
(450) in case at step (440) it is needed to update the values, calculating the value of each i^{th} feature of the new reference point relative to the rest condition (Rext_ref′) as

Rest_ref′(i)=Rest_ref(i)+b·[Rest_2(i)]−Rest_ref(i)]

Wherein b is a coefficient of value between 0 and 1, and preferably between 0.05 and 0.2.
(460) substituting the values of the features relative to the first movement associated with the rest condition individuated at point (410) with the values of the second movement associated with the rest condition individuated at point (420), and starting from point (420) again.

The timer period at point (400) can be modified manually by the user or automatically by the system, in order to compensate possible factors which could alter the features measured during the step of “usage of the prosthetic device”, as for example the variation of the electrode-skin impedance measured directly (through a suitable impedance sensor) or indirectly (through the measure of environmental parameters as temperature and humidity).

The modification of the timer period can be actuated in response to:
i. variations of the value detected by one or more sensors of the system (such as a sensor of the electrode-skin impedance, a sensor of skin temperature or humidity, a sensor for measuring the environmental temperature or humidity, etc.);
ii. variations of the intensity of the activity carried out by the user detected for example by an inertial unit;
iii. after the manual modification provided by the user through a suitable interface with the system (as for example a potentiometer, an ON/OFF button, a toggle button, a graphic interface on dedicated software, etc.);

It is described now the calibration method for controlling the sensors requiring a proportional control of the torque and/or speed, actuated by the prosthesis or orthosis or exoskeleton.

For example, in case of a transradial prosthesis, it is possible to associate the control of the torque and/o speed with the finger closing so that it is proportional to the muscular contraction exerted by the subject.

So, for the proportional control it is needed to derive a command value, to be used for the proportional control of the actuator, from the signals detected by the sensors (electromyography sensors, MMG sensors (mechanomyogram), AMG sensors (acoustic myogram)).

Also in this case, as for the calibration procedure, the procedure can be repeated separately for a plurality of different positions of usage.

It is to be specified that the calibration procedure to manage the proportional control of an actuator is carried out conveniently after the just described supervised calibration procedure, and so, the records of the signals detected in the time domain are present in the memory, and in particular the signals recorded by the sensors at the execution of the movement are present in the memory for which the proportional control is to be calibrated.

The method comprises the steps of:
(1000) defining the mathematical form of a function of dependency of the output value of the proportional control as a function of the features calculated for the signal recorded by each sensor, wherein said output value is normalized in values 0 to 1 (or analogously 0% to 100%).

Said function contains the summation of at least a term directly proportional to each i^{th} feature by means of a respective coefficient (ω1ᵢ). Preferably, said function contains also the summation of at least a term depending exponentially on the value calculated for each i^{th} feature by means of a respective coefficient (ωeᵢ). According to an embodiment said mathematical form can contain at least a term directly proportional to the n^{th} power of the value calculated for the i^{th} feature, by means of a respective coefficient (ωnᵢ). In other terms, the function used is a function of the following kind, in which there are zero, one or more summations next to the first one.

$$V \% = {\sum }_{i = 1}^{K} ω {1}_{i} \cdot Fi + {\sum }_{i = 1}^{K} ω {n}_{i} \cdot {\sum }_{i = 1}^{K} Fi \cdot {e}^{ω {n}_{i}}$$

(1100) setting with value equal to one the values of all the coefficients (ωeᵢ, ωnᵢ) which are not relative to the direct proportionality;
(1200) estimating the values of said coefficients of direct proportionality (ω1ᵢ) by means of an error reduction algorithm which for a plurality of percentage values of the instruction to be used for the actuator imposes that the summation of the maximums recorded during the execution of the movement for each feature (Fi_max), multiplied each by the respective coefficient of proportionality (ω1ᵢ) and by the considered percentage value, is equal to the percentage values of the instruction to be used for the actuator.

Substantially a plurality of relations as the following one are written, each one for a different value of instruction for the actuator

$$V \% = {\sum }_{i = 1}^{K} ω {1}_{i} \cdot V \% \cdot Fi\_max$$

The relations are then used to estimate, according to technique known per se at the state of the art, the values of the coefficients of direct proportionality (ω1ᵢ) which reduce error.

It is to be noted that, for the estimation of the coefficient values, no measured torque value has been used, but only the values deriving from EMG sensors.
(1300) making the user repeat the movement object of the calibration, asking him to increase gradually the torque applied, and providing him with a feedback by means of graphic interface of the applied torque, calculated with the coefficient values estimated at point (1200) and the relation defined at point (1000), in which only the terms relative to the direct proportionality are valued.
(1400) calculating the features of the signals detected by the EMG sensors during step (1300) and estimating the coefficient values of the relation defined at point (1000) according to what described at point (1200) but considering also the exponential terms and/or the ones with proportionality higher than the first one.
(1500) repeating the steps (1300) and (1400) up to when the estimated error goes under a predetermined threshold.

It is to be noted that also in this case no measured torque value has been used, but only the values of EMG signals.

The feedback of point (1300) can be matched and/o substituted with various kinds of proportional feedbacks, as for example:
vibratory/haptic feedback: during the execution of contraction ramps a vibration proportional (in frequency and/or amplitude) to the intensity of the contraction to be developed is exerted through a vibratory motor or a system of vibratory motors;
acoustic feedback: during the execution of contraction ramps it is provided an acoustic signal proportionally modulated in amplitude and/or frequency to the intensity of the muscle contraction to be developed;
visual feedback: further and/or alternative visual feedbacks, as for example:
LED, LED bars or other lighting systems to provide in output a proportional light signal in colour and/or light intensity proportionally modulated to the intensity of the muscle contraction to be developed;
text indications on the same display/monitor used for the calibration steps and/or on an accessory display/monitor so that text indications are provided (through words, absolute or percentage numbers) proportionally modulated to the intensity of the muscle contraction to be developed.

In a further embodiment, the coefficients, feature values, statistical estimators, reference vectors, calibration parameters and/or control parameters calculated during calibration and/or during use of the prosthetic device may be stored over time in a database. Said database may comprise historical data representative of the interaction between the subject and the device, preferably associated with one or more contextual parameters detected during use of the device.

The contextual parameters may comprise, for example, one or more of: temperature, humidity, atmospheric pressure, ambient pressure, skin temperature, residual limb temperature, skin-electrode impedance, electrode contact quality, contact pressure between the device and the subject, socket pressure, sweat or moisture level, acceleration, angular velocity, orientation, vibration, shock, load, force, torque, actuator current, actuator voltage, motor temperature, motor speed, motor position, battery voltage, battery current, battery state of charge, battery state of health, usage time, number of actuation cycles, number of grip cycles, type of movement executed, intensity of the command generated by the subject, speed of execution of the movement, response time of the device, error rate in movement recognition, number of repeated commands, and/or environmental conditions detected by one or more sensors integrated in or connected to the device.

The stored data may be processed to generate statistical information representative of the behaviour of the device, the stability of the calibration, the quality of the signal acquisition, the repeatability of the user commands and/or the evolution over time of the interaction between the subject and the device.

In a further embodiment, the data stored in the database may be processed by means of one or more classification, clustering, regression or anomaly-detection methods in order to classify or estimate an operating condition of the device and/or a condition of use of the device. Said methods may comprise, by way of non-limiting example, threshold-based classification, k-nearest neighbours, support vector machines, decision trees, random forests, Bayesian classifiers, Gaussian mixture models, hidden Markov models, neural networks, clustering algorithms such as k-means or DBSCAN, principal component analysis, regression models, time-series analysis, or combinations thereof.

The classification or estimation may be used to identify one or more conditions selected from: a stable operating condition of the device, a drift of the calibration parameters, a degradation of the quality of the acquired signals, an abnormal variation of the electrode-skin interface, a possible need for recalibration, a possible need for technical assistance, a possible need for replacement of one or more components, a possible need for maintenance, a possible need for software or firmware update, a possible change in the preferred operating mode of the device, or an abnormal or unexpected use condition of the device.

The generated information may be provided to the user, to a clinician, to an orthopaedic technician, to an occupational therapist, to a service technician or to a remote monitoring system. The information may be provided as a warning, recommendation, score, index, trend, classification label, maintenance indication, recalibration indication, or follow-up indication.

In a further embodiment, the variation over time of the calculated coefficients, feature values, statistical estimators, reference vectors and/or control parameters may be used to provide information representative of the ability of the subject to use the device. Said information may comprise, for example, an indication of command repeatability, command stability, separation between different intended movements in the feature space, reduction of unintended activations, reduction of recognition errors, improvement in response time, improvement in proportional control, increase in the number of reliably recognizable movements, or improvement in the consistency of muscle activation patterns.

When the device is configured to operate according to a plurality of operating modes, the stored data may be used to suggest, select or recommend an operating mode suitable for the current ability of the subject. For example, the system may suggest a simpler control mode when the detected signals or command patterns are unstable, and may suggest a more advanced control mode when the detected signals or command patterns are sufficiently stable and repeatable.

In particular, in subjects undergoing rehabilitation, training or recovery following trauma, surgery, amputation or neuromuscular impairment, the stored data may be used to provide the clinician or occupational therapist with information useful for monitoring the evolution of the subject-device interaction over time. Said information may be indicative of a trend of neuromuscular recovery, motor learning, training progression or adaptation to the prosthetic device, said information being intended to support, and not replace, the assessment of the clinician or occupational therapist.

In a further embodiment, the data stored in the database, including the variation over time of the coefficients, feature values, statistical estimators, reference vectors, calibration parameters, control parameters and contextual parameters, may be used to generate training data for mathematical, statistical or machine-learning models, preferably data-driven control models.

Said models may be trained to associate physiological input signals, user commands, contextual parameters and/or device operating parameters with corresponding actuator commands, movement classes, movement trajectories, grip patterns, force levels, torque levels, speed levels or control strategies. The models may comprise, by way of non-limiting example, regression models, classifiers, neural networks, recurrent neural networks, convolutional neural networks, support vector machines, Gaussian mixture models, hidden Markov models, reinforcement learning models, adaptive control models, or hybrid models combining rule-based and data-driven control.

The trained models may be used not only for controlling a prosthetic device, but also for controlling one or more mechanical actuators of an anthropomorphic or non-anthropomorphic mechanical system. For example, the trained models may be used to control an end effector of a robotic arm, a robotic gripper, a robotic hand, an orthotic device, an exoskeleton, a rehabilitation robot, a teleoperated robotic system, an autonomous robotic system, or a collaborative robotic system.

In such embodiments, the data originally acquired during calibration and/or use of the prosthetic device may contribute to the generation of control models capable of translating physiological signals, user intent signals or other command signals into actuator commands suitable for producing coordinated movements of one or more mechanical actuators.

1. A prosthetic device comprising:
a limb or artificial joint or orthosis or a partial or full exoskeleton comprising a mechanism provided with at least an actuator and configured to carry out one or more actions;
a supplying source;
at least an input source modulated by the contraction of one or more muscles of a patient wearing the device, comprising at least an electrode;
electronic computing devices, on which computer programs are loaded, configured to detect signals coming from said at least one input source and to actuate said at least one actuator in order that said mechanism carries out an action as a function of the signals coming from said at least one input source,
and wherein on said electronic computing devices, computer programs are loaded configured to carry out the method comprising the steps of:
(100) recording the signals (Ns) detected by said at least one electrode while said patient wearing the device is in a predetermined condition of movement or rest, for a predetermined time interval ΔT;
(110) subdividing each of said signals (Ns) in a plurality of time intervals of predetermined duration;
(120) extracting from the signal relative to each of the time intervals defined at point (120) at least a characteristic parameter (feature) relative to the signal selected from the list comprising: peak or peak-to-peak average amplitude, peak or peak-to-peak maximum amplitude,
effective value, power spectrum average frequency, power spectrum median frequency, −6 dB power spectrum band, −20 dB power spectrum band;
(130) calculating a statistical estimator for each feature calculated at point (120),
(140) repeating the steps (100) to (130) sequentially with said patient in rest condition and, in the following, with said patient carrying out a known movement selected from a list of a plurality of predetermined movements (Mov_1, Mov_2, . . . , Move_n) for each iteration,
(150) associating a vector defined by the set of the values of the features calculated at point (130) with each movement known carried out by the patient and with the rest condition of point (140), and storing said vectors; and wherein said electronic computing devices are configured to use said stored vectors to generate, during use of the device, control signals for said at least one actuator corresponding to a determined movement of the prosthetic device, as a function of signals detected by said at least one electrode, thereby controlling operation of said actuator in real time.

2. The prosthetic device according to claim 1, wherein said method comprises also a test procedure, comprising the steps of:
(200) detecting the signals detected by said at least one electrode while the user carries out one specific movement,
(210) calculating, with the same modes described in steps (110) to (130), a plurality of statistical estimators, and defining a vector defined by the set of the values of said statistical estimators;
(220) individuating, among all the vectors stored at step (150) and associated with a different movement, the one with the lower Euclidean distance from the point defined at step (210);
(230) providing the user with a feedback indicating the movement individuated at point (220).

3. The prosthetic device according to claim 2, wherein said method provides at step (230) to provide also the user with an indication relative to the Euclidean distance between the vector calculated at step (210) and the vector individuated at step (220).

4. The prosthetic device according to claim 2, wherein said method provides, after step (230), a procedure of usage of said device, comprising the steps of:
(300) acquiring, at predetermined time intervals, the signals (Ns) coming from said at least one electrode, for a predetermined duration and storing them in a relative buffer.
(310) subdividing each of said signals (Ns) in a plurality of time intervals of predetermined duration, thus obtaining “M” signal pieces for each signal.
(320) for each one of the signal piece extracted at step (310) extracting at least a characteristic parameter (feature) according to the same modes followed at step (120).
(330) calculating a statistical estimator of the value of each feature calculated at point (320) according to the same modes followed at step (130) and defining a vector defined by the set of the values of said statistical estimators;
(340) calculating the Euclidean distance between the vector calculated at step (330) and each vector stored at step (150) and associated with a specific movement, thus identifying the movement for which such distance is minimum.
(350) carrying out the movement identified at step (340).

5. The prosthetic device according to claim 4, further comprising a position sensor configured to recognize a plurality of positions of reference and wherein said method provides to repeat the steps (100) to (150) for each one of said positions of reference, storing for each one and for each movement of point (140) as well as for the rest condition a vector defined by the set of the values of the features calculated at point (130).

6. The prosthetic device according to claim 5, wherein said method comprises also, before step (300), the steps of:
(298) acquiring by means of said position sensor the position of the prosthetic device, thus identifying the significant position of reference;
(299) selecting the set of reference points representing each movement relative to the position identified at point (298).

7. The prosthetic device according to claim 1, wherein said method comprises also an automatic updating procedure of the vectors stored at step (150), comprising the steps of:
(400) with the device being used, starting a timer of predetermined period;
(410) storing the values of the features relative to the first movement following the timer expiring of point (400) associated with the rest condition, thus defining a first vector (Rest_1) associated with said first movement associated with the rest condition, and starting a new timer of predetermined period;
(420) storing the values of the features relative to the first movement following the timer expiring of point (410) associated with the rest condition, thus defining a second vector (Rest_2) associated with the rest condition;
(430) calculating the Euclidean distances of each one the two vectors defined at steps (410) and (420) from the reference vector representing the rest condition (Rest_ref) stored at step (150);
(440) in case the distance between the reference vector (Rest_ref) stored at step (150) and said second vector associated with the rest condition defined at step (420) is greater than the distance between the reference vector (Rest_ref) stored at step (150) and said first vector associated with the rest condition defined at step (410), updating the values of said reference vector according to what known at point (450), on the contrary, not updating said values.
(450) in case at step (440) it is needed to update the values, calculating the value of each i^{th} feature of the new reference vector relative to the rest condition (Rext_ref′) as
Rest_ref′(i)=Rest_ref(i)+b·[Rest_2(i)]−Rest_ref(i)]
wherein b is a coefficient of value between 0 and 1,
(460) substituting the values of the features relative to the first movement associated with the rest condition individuated at point (410) with the values of the second movement associated with the rest condition individuated at point (420), and starting from point (420) again.

8. The prosthetic device according to claim 1, further comprising at least an actuator provided with proportional control of the torque and/or speed actuated by the prosthesis or orthosis or exoskeleton, and characterized in that said method comprises the steps of:
(1000) defining the mathematical form of a function of dependency of the output value of said proportional control as a function of the features calculated for the signal recorded by each sensor, said mathematical form comprising the summation of at least a term directly proportional to each i^{th} feature by means of a respective coefficient (ω1i),
(1100) setting with a value equal to 1 the values of all the coefficients (ωei, ωni) defining said mathematical form and which are not relative to the direct proportionality to a feature;
(1200) estimating the values of said coefficients of direct proportionality (ω1i) defined at point (1000) by means of an error reduction algorithm which, for a plurality of percentage values of the instruction to be used for the actuator, imposes that the summation of the maximums recorded during the execution of the movement for each feature (Fi_max), multiplied each by the respective coefficient of proportionality (ω1i) and by the considered percentage value, is equal to the percentage values of the instruction to be used for the actuator.
(1300) making the user repeat the movement object of the calibration, asking him to increase gradually the torque applied, and providing him with a feedback by means of graphic interface of the applied torque, wherein said applied torque is calculated by means of said function defined at point (1000), with the values of the coefficients estimated at point (1200).

9. The prosthetic device according to claim 1, wherein said electronic computing devices are configured to store, over time, in a database, one or more of: coefficients, feature values, statistical estimators, reference vectors, calibration parameters and/or control parameters calculated during calibration and/or during use of the prosthetic device.

10. The prosthetic device according to claim 9, wherein said database further stores one or more contextual parameters detected during use of the device, said contextual parameters comprising one or more of: temperature, humidity, atmospheric pressure, skin temperature, residual limb temperature, skin-electrode impedance, electrode contact quality, contact pressure between the device and the patient, socket pressure, sweat or moisture level, acceleration, angular velocity, orientation, vibration, shock, load, force, torque, actuator current, actuator voltage, motor temperature, motor speed, motor position, battery voltage, battery current, battery state of charge, usage time, number of actuation cycles, type of movement executed, response time of the device and/or error rate in movement recognition.

11. The prosthetic device according to claim 1, wherein said electronic computing devices are configured to process the data stored in said database to generate statistical information representative of the behavior of the device, the stability of the calibration, the quality of the signal acquisition, the repeatability of the user commands and/or the evolution over time of the interaction between the patient and the device.

12. The prosthetic device according to claim 1, wherein said electronic computing devices are configured to process the data stored in said database by means of one or more classification, clustering, regression or anomaly-detection methods in order to classify or estimate an operating condition of the device and/or a condition of use of the device.

13. The prosthetic device according to claim 12, wherein said operating condition or condition of use comprises one or more of: a drift of the calibration parameters, a degradation of the quality of the acquired signals, an abnormal variation of the electrode-skin interface, a possible need for recalibration, a possible need for technical assistance, a possible need for replacement of one or more components, a possible need for maintenance, a possible need for software or firmware update, a possible change in the preferred operating mode of the device, or an abnormal or unexpected use condition of the device.

14. The prosthetic device according to claim 1, wherein said electronic computing devices are configured to process a variation over time of the calculated coefficients, feature values, statistical estimators, reference vectors and/or control parameters to provide information representative of the ability of the patient to use the device and/or to suggest, select or recommend an operating mode suitable for the current ability of the patient.

15. The prosthetic device according to claim 11, wherein the data stored in said database are used to generate training data for mathematical, statistical or machine-learning models, preferably data-driven control models, configured to associate physiological input signals, user commands, contextual parameters and/or device operating parameters with corresponding actuator commands, movement classes, movement trajectories, grip patterns, force levels, torque levels, speed levels or control strategies.
